(19) United States
(12) Patent Application Publication
(10) Pub. No.: US 2024/0197612 A1
(43) Pub. Date: Jun. 20, 2024

(54) COLOURED AQUEOUS DISPERSION OF PARTICLES OF AT LEAST ONE PIGMENT COMPRISING AN IONIC POLYMERIC DISPERSANT, A POLYOL, HYALURONIC ACID OR A SALT THEREOF, A FILLER AND A POLYOXYETHYLENATED ALKYL ETHER DIMETHYLSILANE COMPOUND

(71) Applicant: L'OREAL, Paris (FR)

(72) Inventors: Anthony COLAS, Chevilly Larue (FR); Cécile SAQUET-GOUVILLE, Chevilly Larue (FR)

(21) Appl. No.: 18/553,269

(22) PCT Filed: Mar. 24, 2022
(86) PCT No.: PCT/EP2022/057799

§ 371 Date: Sep. 29, 2023

(30) Foreign Application Priority Data
Apr. 1, 2021 (FR) 2103409

(51) Int. Cl. A61K 8/73; A61K 8/19; A61K 8/29; A61K 8/58; A61K 8/98; A61Q 1/02; A61Q 19/00

(52) U.S. Cl. CPC A61K 8/735; A61K 8/19; A61K 8/29; A61K 8/585; A61K 8/987; A61Q 1/02; A61Q 19/007; A61K 2800/43

(57) ABSTRACT

The present patent application relates to a composition, notably for coating keratin materials, more particularly for making up and/or caring for keratin materials such as the skin, in the form of an aqueous dispersion of at least one pigment, comprising, notably in a physiologically acceptable medium:
a) an aqueous phase; and
b) at least one ionic polymeric dispersant; and
c) at least one hyaluronic acid and/or a salt thereof; and
d) at least one polyol; and
e) at least one polyoxyethylenated alkyl ether dimethylsilane compound; and
f) at least one filler; and
g) at least one pigment.

The present invention is directed towards proposing, for the field of caring for and/or making up keratin materials, notably the skin, a composition in the form of an aqueous dispersion of at least one pigment comprising at least one ionic polymeric dispersant, at least one polyol and at least one hyaluronic acid or a salt thereof.

The invention also relates to a cosmetic process for caring for and/or making up keratin materials, notably the skin, comprising at least the application to said keratin materials of a composition as defined previously.

Cosmetic makeup compositions, for example foundations, are commonly used to give an aesthetic colour to keratin materials such as the skin, but also to enhance the beauty of irregular skin, by making it possible to hide marks and dyschromias, and to reduce the visibility of relief imperfections such as pores and wrinkles. Many formulations have been developed to date. Use is generally made of a large amount of pigments in order to obtain a uniform, covering makeup.

Emulsions are generally appealing to consumers, in particular in the context of foundations, since they are easy to apply. For such compositions, it is sought to provide colour to enhance the beauty of the skin. Substantial coverage is also sought to hide skin defects and to make the complexion uniform. A high proportion of pigments is generally used for this purpose. It is also important for the pigments present in these compositions to have good dispersibility with a view to obtaining a stable and homogeneous composition. Consumers are also looking for products which, after application, afford a light texture with no thickness or lankness effect and which do not transfer onto substrates in contact with the keratin materials such as the skin, for instance clothing.

However, the emulsions, generally water-in-oil emulsions, used in makeup have a tendency to have poor performance as regards the freshness, to produce a greasy effect due to the large presence of oils, generally silicone oils, in these emulsions, and to give a sensation of heaviness of the deposit.

These emulsions also have the drawback of limiting the incorporation of water-soluble active agents in amounts that are efficient to provide one or more care effects such as anti-aging in addition to makeup. Furthermore, they generally require removal with products based on a fatty phase.

Finally, these compositions are not suitable for producing a healthy-complexion effect, which consists in restoring a more uniform, radiant complexion, while at the same time conserving a natural, non-glossy appearance of the skin.

It is known that the skin may lose its vivacity and radiance or may become dull over time, notably due to the effect of ageing, and/or on account of environmental factors such as pollution, the wind or the cold, psychological causes such as fatigue or stress, or on account of hormonal changes such as the menopause. Similarly, certain skin types may have a dull flesh tone with a greyish complexion.

Patent application EP3727298 discloses aqueous dispersions of particles of at least one dyestuff comprising a film-forming polymer in the form of particles in aqueous dispersion, an ionic polymeric dispersant, at least one mattifying filler and at least one thickener for obtaining a matt effect, good persistence of this effect and also a good transfer-resistant effect. However, some of these compositions are not fully satisfactory as regards the sensory properties such as the skin moisturization, the freshness, the light texture without having a thickness or heaviness effect, and as regards the healthy complexion effect and the play time.

There is thus still a need to find novel aqueous formulations for caring for and/or making up keratin materials, in particular the skin, which can overcome the drawbacks mentioned previously.

The Applicant has found, unexpectedly, in the course of its research, that it is possible to achieve this objective with a composition, notably for coating keratin materials, more particularly for making up and/or caring for keratin materials such as the skin, in the form of an aqueous dispersion of at least one pigment, comprising, notably in a physiologically acceptable medium:
a) an aqueous phase; and
b) at least one ionic polymeric dispersant; and
c) at least one hyaluronic acid and/or a salt thereof; and
d) at least one polyol; and
e) at least one polyoxyethylenated alkyl ether dimethylsilane compound; and
f) at least one filler; and
g) at least one pigment.

The compositions according to the invention make it possible to obtain care and/or makeup products which, after application, afford a fresh sensation on the skin, a good moisturizing effect, a light texture with no thickness or heaviness effect and which do not transfer onto substrates in contact with the keratin materials such as the skin, for instance clothing. They are easy to remove with water-based makeup removers. They allow the incorporation of water-soluble active agents in amounts that are efficient for providing one or more care effects in addition to makeup. They are also suitable for giving the skin a healthy complexion effect.

The flesh tone may be defined in the context of the present invention by the phototypes defined in Fitzpatrick's classification (see notably Fitzpatrick, T. B., 1975, “Soleil et peau [Sun and skin]”, Journal de Médecine Esthétique (2): 33-34; Pathak, M. A.; Jimbow, K.; Szabo, G.; Fitzpatrick, T. B. (1976). “Sunlight and melanin pigmentation”. In Smith, K. C. (ed.): Photochemical and Photobiological Reviews, Plenum Press, New York, 1976: 211-239; Fitzpatrick, T. B. (1986). “Ultraviolet-induced pigmentary changes: Benefits and hazards”, Therapeutic Photomedicine, Karger, vol. 15 of “Current Problems in Dermatology”, 1986: 25-38).

The compositions according to the invention are particularly suitable for phototypes I to V, and more particularly for phototypes I to III.

This discovery forms the basis of the invention.

### SUBJECTS OF THE INVENTION

Thus, a first subject of the present invention is a composition, notably for coating keratin materials, more particularly for making up and/or caring for keratin materials such as the skin, in the form of an aqueous dispersion of at least one pigment, comprising, notably in a physiologically acceptable medium:
a) an aqueous phase; and
b) at least one ionic polymeric dispersant; and
c) at least one hyaluronic acid and/or a salt thereof; and
d) at least one polyol; and
e) at least one polyoxyethylenated alkyl ether dimethylsilane compound; and
f) at least one filler; and
g) at least one pigment.

A second subject of the present invention is a process for coating keratin materials, more particularly for making up and/or caring for keratin materials such as the skin, characterized in that it comprises at least the application to the keratin materials of a composition as defined previously.

### Definitions

In the context of the present invention, the term “keratin material” notably means the skin (of the body, face, area around the eyes, or eyelids).

The term “physiologically acceptable” means compatible with the skin and/or its integuments, which has a pleasant colour, odour and feel, and which does not cause any unacceptable discomfort (stinging or tautness) liable to discourage the consumer from using this composition.

The term “aqueous particle dispersion” means any composition comprising an aqueous phase in which are dispersed dyestuff particles that are insoluble in said aqueous phase; said composition not comprising any other phase that is immiscible such as an oily phase in the aqueous phase of the composition, irrespective of the temperature at which the composition is manufactured.

The term “not comprising any immiscible phase” means containing less than 1% by weight of compound(s) that are liquid at room temperature (20-25° C.) and immiscible in the aqueous phase, relative to the total weight of the composition, or even which is free of liquid compound(s) of the composition of the invention.

The term “particle” denotes a solid material that is insoluble in the aqueous phase of the composition, irrespective of the temperature at which the composition is manufactured.

### Aqueous Phase

The composition according to the invention comprises an aqueous phase.

The term “aqueous phase” means a phase comprising water and also all the water-soluble or water-miscible solvents and ingredients.

The aqueous phase may contain a demineralized water or alternatively a floral water such as cornflower water and/or a mineral water such as Vittel water, Lucas water or La Roche Posay water and/or a spring water.

According to a preferential embodiment, the aqueous phase is present in a content ranging from 91% to 95% by weight relative to the total weight of the composition.

### Hyaluronic Acid

The composition according to the present invention contains at least one hyaluronic acid and/or a salt thereof.

The salts may be chosen from the alkali metal salts, and notably the sodium and potassium salts. Sodium hyaluronate is advantageously used.

According to a particular embodiment of the invention, hyaluronic acid and/or a salt thereof may have a weight-average molecular weight ranging from 1 000 000 to 1 700 000 daltons and preferably ranging from 1 000 000 to 1 200 000 daltons.

According to a particular embodiment of the invention, hyaluronic acid and/or a salt thereof may have a number-average molecular weight ranging from 20 000 to 50 000 daltons.

According to a particular embodiment of the invention, use may be made of a mixture of at least two hyaluronic acids of different molecular weights, each of them being in free form or in the form of a salt.

In particular, use will be made of a mixture comprising:
a) at least one hyaluronic compound (A) chosen from hyaluronic acid and/or a salt thereof with a weight-average molecular weight ranging from 1 000 000 to 1 700 000 daltons and preferably ranging from 1 000 000 to 1 200 000 daltons, and
b) at least one hyaluronic compound (B) chosen from hyaluronic acid and/or a salt thereof with a weight-average molecular weight ranging from 20 000 to 50 000 daltons.

According to a preferential form, the weight ratio of hyaluronic compound(s) (B)/hyaluronic compound(s) (A) is greater than 1, more preferentially greater than or equal to 2 and less than 3.

Use may be made, for example, of one or more sodium hyaluronates sold under the following trade names:
Cristalhyal® (weight-average molecular weight 1 000 000-1 700 000 daltons) and Renovhyal LO® (20 000 daltons<number-average molecular weight<50 000 daltons) by the company Soliance;
Bio-Sodium Hyaluronate Powder MMW® (weight-average molecular weight 1 000 000-1 700 000 daltons) by the company Sochibios;
Sodium Hyaluronate HA-T® (weight-average molecular weight 1 000 000-1 700 000 daltons) and Low Molecular Weight Sodium Hyaluronate HA-TLM® (20 000 daltons<number-average molecular weight<50 000 daltons) by the company Bloomage Freda Biopharm;
Sodium Hyaluronate (Cosmetic Grade) (weight-average molecular weight 1 000 000-1 700 000 daltons) by the company Shandong Topscience Biotech.

The hyaluronic acid(s) and/or the salts thereof are preferably present in the composition according to the invention in a total content ranging from 0.01% to 3% by weight, more preferentially ranging from 0.05% to 2% by weight, and more particularly ranging from 0.1% to 1.5% by weight, relative to the total weight of the composition.

### Ionic Polymeric Dispersant

The ionic polymeric dispersants are preferably chosen from:
(meth)acrylic acid homopolymers and salts thereof,
(meth)acrylic acid copolymers and salts thereof,
styrene/(meth)acrylic acid copolymers and salts thereof,
vinylnaphthalene/(meth)acrylic acid copolymers and salts thereof,
styrene/maleic acid copolymers and salts thereof,
vinylnaphthalene/maleic acid copolymers and salts thereof,
maleic anhydride copolymers and salts thereof,
mixtures thereof.

The term “salts” means the salts of an alkali metal such as Na, Li or K of said acids, the salts of monoethanolamine, diethanolamine or triethanolamine or of basic amino acids such as lysine or arginine, of said acids, and mixtures thereof.

Among the maleic anhydride copolymers and salts thereof, mention may be made in particular of the copolymers obtained by copolymerization of one or more maleic anhydride comonomers and of one or more comonomers chosen from vinyl acetate, vinyl alcohol, vinylpyrrolidone, olefins including from 2 to 20 carbon atoms, for instance octadecene, ethylene, isobutylene, diisobutylene, isooctylene, and styrene, and mixtures thereof, the maleic anhydride comonomers being optionally partially or totally hydrolysed, and more particularly copolymers of maleic anhydride and of diisobutylene and salts thereof, and more particularly the product sold under the name Orotan 731DPR by the company Rhodia.

According to a particular embodiment of the invention, the dispersant(s) thus correspond to formula (I) below:

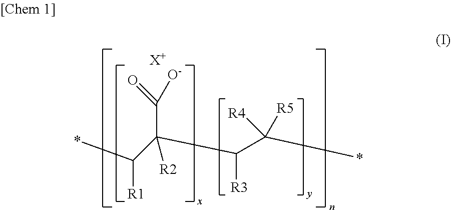

in which:
R1 represents a hydrogen atom, a methyl group or a group *—C(O)O⁻/X⁺ or a group *—C(O)O—R6, with R6 representing a linear or branched C₁-C₅₀, preferably C₁-C₈, preferably C₁-C₄ alkyl group,
R2, R3 and R4, which may be identical or different, representing a hydrogen atom or a methyl group,
R5 representing a phenyl group or a linear or branched C₁-C₅₀, preferably C₁-C₂₀ alkyl group, or a group *—C(O)O—R7, with R7 representing a linear or branched C₁-C₅₀, preferably C₁-C₈, preferably C₁-C₄ alkyl group,
x represents an integer ranging from 1 to 100,
y represents an integer ranging from 0 to 100,
n representing an integer between 1 and 1000,
x, y and n being chosen such that the equation “n (x+y)” is greater than or equal to 20 and preferably greater than or equal to 50,
X⁺ representing a cation notably chosen from a sodium atom, a potassium atom and an ammonium group, and is preferably an ammonium group.

According to a more particular embodiment, the dispersant(s) thus correspond to formula (I′) below:

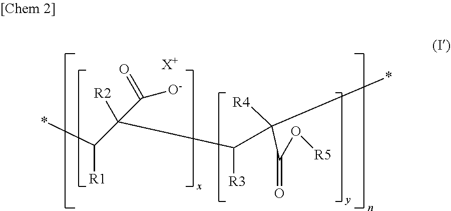

in which:
R1, R2, R3 and R4 represent a hydrogen atom or a C₁-C₆ alkyl group, in particular a methyl,
R5 representing a linear or branched C₁-C₅₀, preferably C₁-C₈, preferably C₁-C₄ alkyl group, in particular a methyl,
x represents an integer ranging from 1 to 100,
y represents an integer ranging from 0 to 100,
n representing an integer between 1 and 1000,
x, y and n being chosen such that the equation “n (x+y)” is greater than or equal to 20 and preferably greater than or equal to 50,
X⁺ representing a cation notably chosen from a sodium atom, a potassium atom and an ammonium group, and is preferably an ammonium group.

According to a more particular embodiment, the dispersant(s) thus correspond to formula (I″) below:

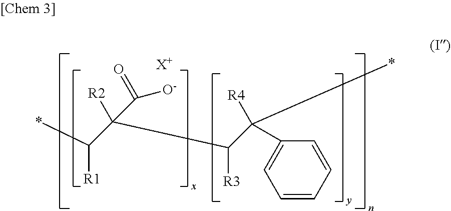

in which:
R1 represents a group *—C(O)O⁻/X⁺ or a group *—C(O)O—R5, with R5 representing a linear or branched C₁-C₅₀, preferably C₁-C₈, preferably C₁-C₄ alkyl group, in particular a methyl,
R2, R3 and R4 representing a hydrogen atom or a C₁-C₆ alkyl group, in particular a methyl,
x represents an integer ranging from 1 to 100,
y represents an integer ranging from 0 to 100,
n representing an integer between 1 and 1000,
x, y and n being chosen such that the equation “n (x+y)” is greater than or equal to 20 and preferably greater than or equal to 50,
X⁺ representing a cation notably chosen from a sodium atom, a potassium atom and an ammonium group, and is preferably an ammonium group.

Preferably, a composition in accordance with the invention comprises at least one ionic polymeric dispersant chosen from:
salts of copolymers of at least two or more monomers chosen from the group comprising acrylic acid, methacrylic acid or an ester thereof,
salts of styrene/maleic anhydride copolymers,
salts of methacrylic acid homopolymer,
and mixtures thereof.

Examples that may be mentioned include the following polymeric dispersants of ionic polymer type:
ammonium salts of a copolymer of at least two or more monomers chosen from the group comprising acrylic acid, methacrylic acid or an ester thereof, of INCI name: Ammonium acrylates copolymer, sold, for example, under the name Syntran KLG219-CG® by the company Interpolymer or Dispex AA 4040® from the company BASF,
ammonium salts of styrene/maleic anhydride (50/50) copolymer, notably in aqueous solution at 30%, sold under the reference SMA 1000H® by the company Arkema,
sodium salts of styrene/maleic anhydride copolymer, notably in aqueous solution at 40%, sold under the reference SMA 1000HNa® by the company Arkema,
a homopolymer of the sodium salt of methacrylic acid of INCI name: Sodium polymethacrylate, notably as an aqueous 25% solution sold under the reference Darvan® 7-N by the company R.T. Vanderbilt Company, Inc.,
mixture(s) thereof.

Use will be made more particularly of a polymer of the sodium salt of methacrylic acid of INCI name: Sodium polymethacrylate, notably as an aqueous 25% solution sold under the reference Darvan® 7-N by the company R.T. Vanderbilt Company, Inc.

A composition according to the invention preferably includes a total content of ionic polymeric dispersant active material of greater than or equal to 10% by weight, in particular ranging from 10% to 30% by weight, relative to the total weight of the composition, more particularly ranging from 10% to 20% by weight, relative to the total weight of the composition.

### Polyol

The composition also comprises a polyol that is miscible with water at room temperature (25° C.) notably chosen from polyols containing from 2 to 20 carbon atoms, preferably containing from 2 to 10 carbon atoms and preferentially containing from 2 to 8 carbon atoms, such as glycerol, propylene glycol, 1,3-butylene glycol, pentylene glycol, hexylene glycol, caprylyl glycol, dipropylene glycol or diethylene glycol; glycol ethers (notably containing from 3 to 16 carbon atoms) such as mono-, di- or tripropylene glycol (C₁-C₄)alkyl ethers, mono-, di- or triethylene glycol (C₁-C₄)alkyl ethers; polyethylene glycols; and mixtures thereof.

Use will more preferentially be made of glycerol, propylene glycol or mixtures thereof, and more particularly a mixture of glycerol and propylene glycol.

According to a preferential embodiment of the invention, the polyol(s) are present in the composition according to the invention in a total content≥5%, preferably ranging from 5% to 15% by weight and more preferentially ranging from 5% to 10% by weight relative to the total weight of the composition.

### Pigments

The composition according to the invention comprises at least one pigment.

The term “pigments” means white or coloured, mineral or organic particles, which are insoluble in an aqueous medium, and which are intended to colour and/or opacify the resulting composition and/or deposit. These pigments may be white or coloured, and mineral and/or organic.

Preferably, the composition comprises up to 10.0% by weight of pigments relative to the total amount, more preferentially from 2% to 8% by weight of pigments, in particular from 4% to 6% by weight relative to the total weight of said composition.

According to a particular embodiment, the pigments used according to the invention are chosen from mineral pigments.

The term “mineral pigment” means any pigment that satisfies the definition in Ullmann's encyclopedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment that is useful in the context of the present invention is generally greater than 100 nm and may range up to 10 μm, preferably from 200 nm to 5 μm and more preferentially from 300 nm to 1 μm.

According to a particular form of the invention, the pigments have a size characterized by a D[50] greater than 100 nm and possibly ranging up to 10 μm, preferably from 200 nm to 5 μm and more preferentially from 300 nm to 1 μm.

The sizes are measured by static light scattering using a commercial MasterSizer 3000 particle size analyser from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is notably described in the publication by Van de Hulst, H. C., Light Scattering by Small Particles, Chapters 9 and 10, Wiley, New York, 1957.

D[50] represents the maximum size exhibited by 50% by volume of the particles.

The mineral pigment(s) are preferably chosen from titanium dioxides, iron oxides and nacres, and mixtures thereof.

In the context of the present invention, an example of a titanium dioxide having the INCI name Titanium Dioxide that may be mentioned is the product sold under the trade name Hombitan FF Pharma® by the company Venator;

In the context of the present invention, examples of iron oxides having the INCI name Iron Oxides (CI: 79499, 79491, 79492) that may be mentioned include the products sold under the trade names Sunpuro Black Iron Oxide C33-7001®; Sunpuro Red Iron Oxide C33-8001®; Sunpuro Red Iron Oxide C33-8001® by the company Sun.

As mineral pigments that may be used in the invention, mention may also be made of nacres.

### Nacres

The term “nacre” should be understood as meaning coloured particles of any form, which may or may not be iridescent, notably produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica notably coated with an organic dye, and also nacreous pigments based on bismuth oxychloride.

They may also be mica particles, at the surface of which are superposed at least two successive layers of metal oxides and/or of organic dyestuffs.

These particles may be chosen from particles containing a natural or synthetic substrate at least partially coated with at least one layer of at least one metal oxide, chosen, for example, from titanium oxides, notably TiO₂, iron oxides, notably Fe₂O₃, tin oxides, chromium oxides, barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

Examples of such particles that may be mentioned include particles including a synthetic mica substrate coated with titanium dioxide, or glass particles, in particular calcium borosilicate particles, coated either with brown iron oxide, with titanium oxide, with tin oxide or with a mixture thereof, for instance the particles sold under the brand name Reflecks® by the company Engelhard.

The nacres may have a yellow, pink, red, bronze, orange, brown, gold and/or coppery tint.

Non-limiting examples of nacres that may be mentioned in particular, alone or as a mixture, include nacres including the following elements:
mica-titanium oxide (for example sold under the commercial reference Timica Sparkle 110P® from BASF, Flamenco Blue® from BASF, Candurin Brown Amber® and Timiron Supersilk MP 1005® from Merck, and Pearlescent Pigment Prestige Gold® from Sudarshan);
mica-titanium oxide coated with N-lauroyl-L-lysine (for example sold under the commercial reference LLD-10 Flamenco Violet® from Daito Kasei Kogyo);
mica-iron oxide (for example sold under the commercial reference Pearlescent Pigment Prestige Soft Bronze® from Sudarshan, Colorona Bronze Sparkle® from Merck, Timica Golden Bronze® from BASF, Candurin Light Gold® from Merck, Duocrome YR 422 C® from BASF, Gemtone Tan Opal G 005® from BASF);
mica-titanium oxide-iron oxide (for example sold under the commercial reference Timica Brilliant Gold 212 G Gemtone Goldstone G0014®, Gemtone Tan Opal G 005® or Timica Golden Bronze® from BASF);
mica-bismuth oxychloride-iron oxide (for example sold under the commercial reference Chroma-Lite Yellow® from BASF);
mica-titanium oxide-chromium oxide-iron oxide (for example sold under the commercial reference Gemtone Jade® from BASF);
mica-titanium oxide-tin oxide (for example sold under the commercial reference Flamenco Super Blue 630 Z® from BASF, Helios R100R® from Topy);
mica-silica-titanium oxide (for example sold under the commercial reference Timiron Splendid Red® from Merck);
platelets of sodium calcium borosilicate coated with titanium oxide and tin oxide (for example sold under the commercial reference Reflecks Rays of Red G430L® from BASF);
platelets of sodium calcium borosilicate coated with tin oxide and iron oxide (for example sold under the commercial reference Mirage Sparkling Champagne® from Eckart);
platelets of calcium aluminium borosilicate coated with titanium oxide and tin oxide, optionally treated with silica (for example sold under the commercial reference Ronastar® from Merck);
platelets of calcium aluminium borosilicate coated with silicon oxide and tin oxide (for example sold under the commercial reference Ronastar Golden Jewel SQ® from Merck);
glass powder coated with titanium oxide (for example sold under the commercial reference Reflecks Dimensions Glittering Gold G230S® from BASF or Metashine MC1120RY® from NSG);
alumina and titanium oxide (for example sold under the commercial reference Spectraflex Focus Red C88-1031® from Sun);
bronze powder (for example sold under the commercial reference Visionaire Bright Sunflower Gold® from Eckart);
synthetic fluorophlogopite and magnesium silicate and titanium dioxide and tin oxide (for example sold under the commercial reference Sunshine Daybreak Blue® from Sun Chemical).

For the purposes of the present invention, the term “mica” includes natural micas and synthetic micas, for instance fluorphlogopites.

The pigments may also be organic pigments.

The term “organic pigment” refers to any pigment that satisfies the definition in Ullmann's encyclopedia in the chapter on organic pigments. The organic pigment may notably be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane and quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

These pigments may also be in the form of composite pigments as described in patent EP 1 184 426. These composite pigments may notably be composed of particles including a mineral core at least partially covered with an organic pigment and at least one binder for fixing the organic pigments to the core.

The pigment may also be a lake. The term “lake” means insolubilized dyes adsorbed onto insoluble particles, the assembly thus obtained remaining insoluble during use.

The mineral substrates onto which the dyes are adsorbed are, for example, alumina, silica, calcium sodium borosilicate or calcium aluminium borosilicate and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090).

An example of a lake that may be mentioned is the product known under the name D&C Red 7 (CI 15 850:1).

### Polyoxyethylenated Alkyl Ether Dimethylsilane Compound

The composition of the invention comprises at least one polyoxyethylenated alkyl ether dimethylsilane compound in which the alkyl radical is a saturated, linear or branched C₁-C₆ alkyl, preferably methyl.

In particular, use may be made of the compound having the INCI name Bis-PEG-18 Methyl Ether Dimethylsilane, such as the commercial product sold under the name Dowsil 2511 Cosmetic Wax® by the company Dow Corning.

The polyoxyethylenated alkyl ether dimethylsilane compound is preferably present in the composition according to the invention in a content ranging from 1.5% to 6% by weight, and more preferentially from 1.9% to 2.7% by weight, relative to the total weight of the composition.

### Fillers

The compositions in accordance with the invention comprise at least one filler, of organic or mineral nature, notably making it possible to give them additional properties in terms of improved sensory quality, play time, mattness, coverage, persistence and/or stability.

The term “filler” should be understood as meaning colourless or white solid particles of any shape, which are in a form that is insoluble and dispersed in the medium of the composition. These particles, of mineral or organic nature, give body or rigidity to the composition and/or softness and uniformity to the makeup.

The fillers used in the compositions according to the present invention may be in lamellar, globular or spherical form, in the form of fibres or in any other intermediate form between these defined forms.

The fillers according to the invention may or may not be surface-coated, and in particular they may be surface-treated with silicones, amino acids, fluoro derivatives or any other substance that promotes the dispersion and compatibility of the filler in the composition.

Examples of mineral fillers that may be mentioned include clays, talc, mica, silica, hollow silica microspheres, kaolin, calcium carbonate, magnesium carbonate, hydroxyapatite, boron nitride, glass or ceramic microcapsules, composites of silica and of titanium dioxide, such as the series TSG® sold by Nippon Sheet Glass, hydrophobic silica aerogel particles surface-modified with trimethylsilyl groups, for example the aerogel sold under the name VM-2260® (INCI name: Silica Silylate) by the company Dow Corning, the particles of which have an average size of about 1000 microns and a specific surface area per unit mass ranging from 600 to 800 m²/g, lipophilic clays, and mixtures thereof.

The clays may be natural or synthetic, and they are made lipophilic by treatment with an alkylammonium salt such as a C₁₀ to C₂₂ ammonium chloride, for example distearyldimethylammonium chloride.

They may be chosen from bentonites, in particular hectorites and montmorillonites, beidellites, saponites, nontronites, sepiolites, biotites, attapulgites, vermiculites and zeolites.

They are preferably chosen from hectorites.

Use will be made in particular of a hectorite modified with distearyldimethylammonium chloride, for instance the commercial product sold under the name Bentone Gel ISD V® by the company Elementis.

Examples of organic fillers that may be mentioned include polyamide powders (Nylon® Orgasol from Atochem), polyethylene powders, polymethyl methacrylate powders, polytetrafluoroethylene (Teflon®) powders, acrylic acid copolymer powders (Polytrap® from the company Dow Corning), lauroyllysine, hollow polymer microspheres such as those of polyvinylidene chloride/acrylonitrile, for instance Expancel® (Nobel Industrie), polyurethane powders, in particular hexamethylene diisocyanate/trimethylol hexyl lactone copolymer powder which are notably commercially available, for example under the name Plastic Powder D-400® or Plastic Powder D-8000 from the company Toshiki, silicone resin microbeads (for example Tospearls® from Toshiba), synthetic or natural micronized waxes, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, Polypore® L 200 (Chemdal Corporation), crosslinked elastomeric organopolysiloxane powders coated with silicone resin, notably with silsesquioxane resin, as described, for example, in patent U.S. Pat. No. 5,538,793, and mixtures thereof. The filler may also be cellulose powder, for instance the product sold under the trade name Tego Feel C 10® by the company Evonik Goldschmidt.

Use will be made more particularly of a cellulose powder, for instance the product sold under the trade name Tego Feel C 10® by the company Evonik Goldschmidt.

The filler(s) are preferably present in the composition according to the invention in a total content ranging from 0.5% to 2% by weight and more preferentially ranging from 0.5% to 0.8% by weight relative to the total weight of the composition.

### Additives

The compositions according to the invention may also contain additional cosmetic ingredients conventionally used for the formulation of particular presentation forms, generally adjusted with regard to the intended keratin material. This or these additional cosmetic ingredients may notably be chosen from hydrophilic thickeners; water-soluble active agents other than hyaluronic acid and salts thereof; pH modifiers such as lactic acid; buffers such as hydroxyethylpiperazineethanesulfonic acid; chelating agents such as ethylenediaminetetraacetic acid, ethylenediaminedisuccinic acid or a salt thereof; preserving agents; antioxidants; fragrances; water-soluble organic UV-screening agents; mineral UV-screening agents; bactericides; odour absorbers, and mixtures thereof.

The amounts of the additional cosmetic ingredients are those conventionally used in the field under consideration, for example from 6% to 20% of the total weight of the composition and preferably from 6% to 10% of the total weight of the composition.

Needless to say, a person skilled in the art will take care to select the optional additional ingredients and/or the amount thereof such that the advantageous properties of the composition according to the invention are not, or are not substantially, adversely affected by the envisaged addition.

### Active Agents

The active agents may be chosen from:
water-soluble vitamins, for instance vitamin B5 and derivatives thereof, notably the salt calcium pantothenate, such as the commercial products sold under the name Calcium D Pantothenate® (0412678) by the company DSM Nutritional Products, D-Calcium Pantothenate USP® by the company BASF; vitamin C and derivatives thereof such as 2-o-alpha-d-glucopyranosyl-L-ascorbic acid having the INCI name Ascorbyl Glucoside, such as the commercial products sold under the names V2G® by the company Macrocare and AA2G® by the company Hayashibara;
anti-ageing agents, such as the rye seed extracts having the INCI name Secale cereale (Rye) Seed Extract, for instance the commercial product sold under the name Coheliss GR® by the company Silab; peptides such as the compound having the INCI name Dipeptide Diaminobutyroyl Benzylamide Diacetate, for instance the commercial product sold under the name SYN-AKE® by the company DSM; the juice of the black crowberry fruit from Lapland having the INCI name Empetrum nigrum Fruit Juice, for instance the commercial product sold under the name Camaderm® by the company Solabia;
mixtures thereof.

### Additional Dyestuffs

A composition according to the invention may also comprise at least one additional dyestuff, preferably in a proportion of at least 0.01% by weight relative to the total weight of the composition.

For obvious reasons, this amount is liable to vary significantly with regard to the intensity of the desired colour effect and of the colour intensity afforded by the dyestuffs under consideration, and its adjustment clearly falls within the competence of a person skilled in the art.

The additional dyestuffs that are suitable for use in the invention may be water-soluble.

For the purposes of the invention, the term “water-soluble dyestuff” means any natural or synthetic, generally organic compound, which is soluble in an aqueous phase or water-miscible solvents and which is capable of imparting colour.

As water-soluble dyes that are suitable for use in the invention, mention may be made notably of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophyllin, methylene blue, anthocyanins (enocianin, black carrot, hibiscus, elder), caramel and riboflavin.

The water-soluble dyes are, for example, beetroot juice and caramel.

### Cosmetic Compositions

The present invention also relates to a cosmetic composition comprising, in a physiologically acceptable medium, a composition as defined above.

The term “physiologically acceptable” means compatible with the skin and/or its integuments, which has a pleasant colour, odour and feel, and which does not cause any unacceptable discomfort (stinging or tautness) liable to discourage the consumer from using this composition.

The physiologically acceptable medium is generally adapted to the nature of the support onto which the composition is to be applied, and also to the form in which the composition is to be packaged.

According to a particularly preferred form, the compositions according to the invention are in very fluid form, notably in the form of a serum having a dynamic viscosity at 25° C. preferably ranging from 50 to 200 centipoises (50 to 130 mPa·s) and more particularly from 90 to 170 poises (90 to 170 mPa·s).

The viscosity of the compositions according to the invention may notably be measured by means of a rotating-spindle rheometer such as the Rheomat RM 200® machine sold by the company Lamy, this machine being equipped with a different spindle (MS-R1, MS-R2, MS-R3, MS-R4 or MS-R5) depending on the viscosity, rotating at 200 rpm. The compositions according to the invention advantageously have a viscosity, both after 30 seconds and after 10 minutes of shear, as defined above.

### Applications

According to one embodiment, a composition of the invention may advantageously be in the form of a composition for caring for the skin, notably of the body or the face, in particular the face, for instance a moisturizing, anti-ageing or flesh-plumping product.

According to another embodiment, a composition of the invention may advantageously be in the form of a composition for making up keratin materials, in particular the skin of the body or of the face, in particular of the face, notably a foundation.

According to another embodiment, a composition of the invention may advantageously be in the form of a composition for making up and caring for keratin materials, in particular the skin of the body or of the face, in particular of the face, notably a moisturizing and flesh-plumping foundation.

Such compositions are notably prepared according to the general knowledge of a person skilled in the art.

Throughout the description, including the claims, the term “including a” should be understood as being synonymous with “including at least one”, unless otherwise specified.

### EXAMPLE 1: MOISTURIZING AND FLESH-PLUMPING TINTED SERUM

TABLE 1

| | | | Example | Example | Example |
|---|---|---|---|---|---|
| | | | 1a outside | 1b outside | 1c outside |
| | | Example 1 | the | the | the |
| Phase | Ingredients (INCI name) | invention | invention | invention | invention |
| A | Water | 37.9 | 37.9 | 37.9 | 37.9 |
| | Caprylyl glycol | 0.3 | 0.3 | 0.3 | 0.3 |
| | Chlorphenesin | 0.2 | 0.2 | 0.2 | 0.2 |
| | Glycerol | 5.0 | 5.0 | 5.0 | 5.0 |
| | Propylene glycol | 2.6 | 2.6 | 2.6 | 2.6 |
| | Calcium pantothenate | 0.4 | 0.4 | 0.4 | 0.4 |
| | PEG-60 Hydrogenated | 0.2 | 0.2 | 0.2 | 0.2 |
| | castor oil | | | | |
| | Bis-PEG-18 methyl ether | 2.3 | 0 | 2.3 | 0 |
| | dimethylsilane | | | | |
| | (Dowsil 2511 Cosmetic | | | | |
| | Wax ® from Dow Corning) | | | | |
| C | Phenoxyethanol | 0.7 | 0.7 | 0.7 | 0.7 |
| | Hydroxyethylpiperazineethanesulfonic | 2.0 | 2.0 | 2.0 | 2.0 |
| | acid | | | | |
| | Ascorbyl glucoside | 0.2 | 0.2 | 0.2 | 0.2 |
| | Empetrum nigrum fruit | 0.1 | 0.1 | 0.1 | 0.1 |
| | juice | | | | |
| | Trisodium | 0.2 | 0.2 | 0.2 | 0.2 |
| | ethylenediaminedisuccinate | | | | |
| | Secale cereale (rye) seed | 0.1 | 0.1 | 0.1 | 0.1 |
| | extract | | | | |
| | Dipeptide diaminobutyroyl | 0.4 | 0.4 | 0.4 | 0.4 |
| | benzylamide diacetate | | | | |
| | Sodium polymethacrylate | 3.4 | 3.4 | 3.4 | 3.4 |
| | (Darvan-7N ® from R.T. | | | | |
| | Vanderbilt Company, Inc.) | | | | |
| | Dimethicone (and) | 0.3 | 0.3 | 0.3 | 0.3 |
| | polysorbate 65 (and) | | | | |
| | simethicone | | | | |
| B | Sodium hyaluronate | 0.3 | 0.3 | 0 | 0 |
| | M.W. = 1 100 000 | | | | |
| | (Cristalhyal LO ® from | | | | |
| | Soliance) | | | | |
| | Sodium hyaluronate 20 000 | 0.7 | 0.7 | 0 | 0 |
| | Da < M.W. < 50 000 Da | | | | |
| | (Renovhyal LO ® from | | | | |
| | Soliance) | | | | |
| D | Water | qs 100 | qs 100 | qs 100 | qs 100 |
| E | Lactic acid pH 5.4-6 | 0.3 | 0.3 | 0.3 | 0.3 |
| A2 | Cellulose (Tego Feel C 10 ® | 0.8 | 0 | 0.8 | 0 |
| | from Evonik Goldschmidt) | | | | |
| A3 | Synthetic fluorophlogopite | 0.5 | 0.5 | 0.5 | 0.5 |
| | Titanium dioxide (CI: | 2.9 | 2.9 | 2.9 | 2.9 |
| | 77891) | | | | |
| | Yellow iron oxides (CI: | 0.8 | 0.8 | 0.8 | 0.8 |
| | 77492) | | | | |
| | Red iron oxides (CI: 77491) | 0.2 | 0.2 | 0.2 | 0.2 |
| | Black iron oxides (CI: | 0.1 | 0.1 | 0.1 | 0.1 |
| | 77499 ) | | | | |
| A4 | Mica (and) iron oxides | 1.0 | 1.0 | 1.0 | 1.0 |
| | (and) titanium dioxide/ | | | | |
| | mica (and) CI 77491 (and) | | | | |
| | CI 77891 | | | | |
| | (Gemtone Tan Opal G | | | | |
| | 005 ® from BASF) | | | | |
| | Mica (and) titanium dioxide/ | 1.0 | 1.0 | 1.0 | 1.0 |
| | mica (and) CI 77891 | | | | |
| | (Timiron Supersilk MP | | | | |
| | 1005 ® from Merck) | | | | |

### Protocol for Preparing the Composition

### Step 1: Preparation of the White Base

The ingredients of phase A were prepared and mixed at room temperature (25° C.) and then heated to 50° C. The ingredients of phase B were added and mixed at room temperature. Once the gel texture was obtained, phase C was added at room temperature. Phase D was then added to dilute the mixture. The pH was then adjusted by adding phase E.

### Step 2: Preparation of the Tinted Serum

Phases A2, A3 and A4 were added to the white base at room temperature and in this order, followed by mixing until a homogeneous aqueous dispersion was obtained.

### Test for Measuring the Spreading of the Film

The following protocol was repeated three times
Uptake of 1 g of each formulation
Application to a glass plate, starting from the same baseline
Spreading to a thickness of 20 μm using a Braive® film spreader reference 354001081
Measurement of the distance from the end of the film to the baseline, in cm
Calculation of the mean spreading EM of the film over three measurements
Calculation of the variation in spreading relative to that of Example 1 according to the following equation: (E_{M1}−E_{M1i})/E_{M1} where E_{M1} is the mean spreading of Example 1 according to the invention and Emi, is the mean spreading of Example 1a, 1b or 1c outside the invention.

The results obtained are collated in the following table.

TABLE 2

| | | Example 1a | Example 1b | Example 1c |
|---|---|---|---|---|
| | Example 1 | outside the | outside the | outside the |
| Formulations | invention | invention | invention | invention |
| % variation in | — | −23.6% ± | −25.1% ± | −65.5% ± |
| spreading relative | | 1.7% | 11.4% | 5.3% |
| to Example 1 of | | | | |
| the invention | | | | |

The results showed that the ternary combination of the sodium salts of hyaluronic acid, of the polyoxyethylenated alkyl ether dimethylsilane compound and of the cellulose filler in Example 1 of the invention leads to better spreading than that of Examples 1a, 1b and 1c outside the invention including only one of these components, two of these components or none of these components.

### Test for Measuring the Film Homogeneity

An application card (reference BYK Byko-Chart Reorder® 2814) was placed on the vacuum table and the vacuum pump was switched on.
The 20 μm-edge applicator (Braive 354001081 film spreader) was positioned in front of the attachment bar
using a pipette, bulk was taken up and an appropriate amount of product was placed in front of the applicator
the “advance” (10 mm/sec) button was pressed to start the application
at the end, the applicator was stopped automatically
the applicator was removed for cleaning, and the “return” button was pressed
once the chariot had returned to its starting position, the applied card was removed and the homogeneity was evaluated.

The homogeneity of the film obtained was scored in the following manner:
O homogeneous makeup result with little presence of traces and of white zone that are difficult to see with the naked eye
I heterogeneous makeup result with presence of traces and of white zone that are visible to the naked eye
II heterogeneous makeup result with pronounced presence of traces and of white zone
III heterogeneous makeup result with very strong presence of traces and of white zone

The results obtained are collated in the following table.

TABLE 3

| | | Example 1a | Example 1b | Example 1c |
|---|---|---|---|---|
| | Example 1 | outside the | outside the | outside the |
| Formulations | invention | invention | invention | invention |
| Appearance of the | O | I | II | III |
| film | | | | |

The results showed that the ternary combination of the sodium salts of hyaluronic acid, of the polyoxyethylenated alkyl ether dimethylsilane compound and of the cellulose filler in Example 1 of the invention leads to better spreading than that of Examples 1a, 1b and 1c outside the invention including only one of these components, two of these components or none of these components.

The results showed that the ternary combination of the sodium salts of hyaluronic acid, of the polyoxyethylenated alkyl ether dimethylsilane compound and of the cellulose filler in Example 1 of the invention led to a uniform makeup result with presence of traces and of white zone that are difficult to see with the naked eye, in contrast with Examples 1a, 1b and 1c outside the invention including only one of these components, two of these components or none of these components.

1. Composition in the form of an aqueous dispersion of at least one pigment, comprising, in a physiologically acceptable medium:
a) an aqueous phase; and
b) at least one ionic polymeric dispersant;
c) at least one hyaluronic acid and/or a salt thereof;
d) at least one polyol;
e) at least one polyoxyethylenated alkyl ether dimethylsilane compound;
f) at least one filler; and
g) at least one pigment.

2. Composition according to claim 1, in which the aqueous phase is present in a content ranging from 91% to 95% by weight relative to the total weight of the composition.

3. Composition according to claim 1, in which the at least one ionic polymeric dispersant is chosen from:
(meth)acrylic acid homopolymers and salts thereof,
(meth)acrylic acid copolymers and salts thereof,
styrene/(meth)acrylic acid copolymers and salts thereof,
vinylnaphthalene/acrylic acid copolymers and salts thereof,
styrene/maleic acid copolymers and salts thereof,
vinylnaphthalene/maleic acid copolymers and salts thereof,
copolymers of maleic anhydride and of diisobutylene, and salts thereof, and
mixtures thereof.

4. Composition according to claim 1, in which the at least one ionic polymeric dispersant is chosen from:
salts of copolymers of at least two or more monomers chosen from the group comprising acrylic acid, methacrylic acid or an ester thereof,
salts of styrene/maleic anhydride copolymers,
salts of methacrylic acid homopolymer, and
mixtures thereof.

5. Composition according to claim 1, including a total content of ionic polymeric dispersant active material of greater than or equal to 10% by weight relative to the total weight of the composition.

6. Composition according to claim 1, wherein the at least one hyaluronic acid and/or a salt thereof is at least one sodium hyaluronate.

7. Composition according to claim 1, in which the at least one hyaluronic acid and/or a salt thereof has a weight-average molecular weight ranging from 1 000 000 to 1 700 000 daltons.

8. Composition according to claim 1, in which the at least one hyaluronic acid and/or a salt thereof has a number-average molecular weight ranging from 20 000 to 50 000 daltons.

9. Composition according to claim 1, wherein the at least one hyaluronic acid and/or a salt thereof is at least two hyaluronic acids of different molecular weights, each of them being in free form or in the form of a salt.

10. Composition according to claim 9, comprising:
a) at least one hyaluronic compound (A) chosen from hyaluronic acid and/or a salt thereof with a weight-average molecular weight ranging from 1 000 000 to 1 700 000 daltons, and
b) at least one hyaluronic compound (B) chosen from hyaluronic acid and/or a salt thereof with a weight-average molecular weight ranging from 20 000 to 50 000 daltons.

11. Composition according to claim 10, in which weight ratio of hyaluronic compound(s) (B)/hyaluronic compound(s) (A) is greater than 1.

12. Composition according to claim 1, in which the at least one hyaluronic acid and/or salts thereof are present in a total content ranging from 0.01% to 3% by weight relative to the total weight of the composition.

13. Composition according to claim 1, in which the at least one polyol is chosen from those containing from 2 to 20 carbon atoms.

14. Composition according to claim 1, in which the at least one polyol is chosen from glycerol, propylene glycol, and mixtures thereof.

15. Composition according to claim 1, in which the at least one polyol is present in a total content≥5% weight, relative to the total weight of the composition.

16. Composition according to claim 1, comprising up to 10.0% by weight of the at least one pigment relative to the total weight of said composition.

17. Composition according to claim 1, in which the at least one pigment is at least one mineral pigment and the at least one mineral pigment is chosen from titanium dioxides, iron oxides, nacres, and mixtures thereof.

18. Composition according to claim 1, in which the at least one polyoxyethylenated alkyl ether dimethylsilane compound includes a saturated, linear or branched C₁-C₆ alkyl group.

19. Composition according to claim 1, in which the at least one polyoxyethylenated alkyl ether dimethylsilane compound is present in a content ranging from 1.5% to 6% by weight relative to the total weight of the composition.

20. Composition according to claim 1, in which the at least one filler is present in a content ranging from 0.5% to 2% by weight relative to the total weight of the composition.

21. Composition according to claim 1, in which the at least one filler is at least one cellulose powder.

22. Composition according to claim 1, having a dynamic viscosity at 25° C. from 50 to 200 centipoises (50 to 130 mPa·s).

23. Process for coating a keratin material, comprising applying to the keratin material a composition according to claim 1.
